(19) United States
(12) Patent Application Publication
(10) Pub. No.: US 2009/0117105 A1
(43) Pub. Date: May 7, 2009

(54) HUMANIZED ANTI-VENEZUELAN EQUINE ENCEPHALITIS VIRUS RECOMBINANT ANTIBODY

(75) Inventors: Wei-Gang Hu, Medicine Hat (CA); Leslie P. Nagata, Medicine Hat (CA)

Correspondence Address: BLAKE, CASSELS & GRAYDON LLP, BOX 25, COMMERCE COURT WEST, 199 BAY STREET, SUITE 2800, TORONTO, ON, M5L 1A9, CA

(73) Assignee: Her Majesty The Queen in Right of Canada as Represented by the Minister of National Defence, Ottawa (CA)

(21) Appl. No.: 11/933,948

(22) Filed: Nov. 1, 2007

(51) Int. Cl. A61K 39/395; C07H 21/04; C07K 16/08; C12N 1/21; C12N 15/00

(52) U.S. Cl. 424/133.1; 435/252.33; 435/320.1; 530/387.3; 536/23.53

(57) ABSTRACT

A CDR grafted humanized rAb comprises a human Ig framework having CDRs from murine mAb 1A4A1 VH and VL. DNA sequences and vectors incorporating such sequences are also provided as are pharmaceutical preparations and methods of using the humanized rAbs.

### FIELD OF THE INVENTION

The present invention relates to a humanized antibody (Ab) and, more specifically, to a humanized recombinant Ab (rAb) directed to the Venezuelan equine encephalitis virus (VEEV).

### BACKGROUND OF THE INVENTION

Venezuelan equine encephalitis virus (VEEV), a member of the alphavirus genus of the family Togaviridae, is an important mosquito-borne pathogen in humans and equides [1]. VEEV infections mainly target the central nervous system and lymphoid tissues causing severe encephalitis in equines and a spectrum of human diseases ranging from unapparent or sub-clinical infection to acute encephalitis. Neurological disease appears in 4-14% of cases. The incidence of human infection during equine epizootics could be up to 30%. Mortality associated with the encephalitis in children is as high as 35%. Recent outbreaks in Venezuela and Colombia in 1995 resulted in around 100,000 human cases with more than 300 fatal encephalitis cases [2]. Furthermore, VEEV is highly infectious by aerosol inhalation in humans and other animals. However, there are no antiviral drugs available that are effective against VEEV although currently there are two forms of IND (investigational new drug) VEEV vaccines available for human and veterinary use: TC-83, a live-attenuated Trinidad donkey strain and C-84, a formalin-inactivated TC-83 [3,4]. However, for various reasons, these vaccines are far from satisfactory. For example, approximately 20% of recipients that receive the TC-83 vaccine fail to develop neutralizing Abs, while another 20% exhibit reactogenicity. In addition, the TC-83 vaccine could revert to wild-type form. The vaccine C-84 is well tolerated, but requires multiple immunizations, periodic boosts, and fails to provide protection against aerosol challenge in some rodent models.

Like the other alphaviruses, VEEV is an enveloped virus, consisting of three structural proteins: a capsid encapsidating the viral RNA genome, and two envelope glycoproteins, E1 and E2. E1 and E2 form heterodimers, which project from the virus envelope as trimer spikes. Epitopes on the spikes are the targets of neutralizing Abs. Studies have shown that the viral neutralizing epitopes are mainly located on the E2 protein, and that the E2^{C} epitope appears to be the hub of the neutralization epitopes [5,6]. The murine monoclonal Ab (mAb) 1A1A4 [14] is specific for E2^{C}. This mAb has been shown to be efficient in protecting animals from a lethal peripheral challenge with virulent VEEV [7].

Murine mAbs, however, have serious disadvantages as therapeutic agents in humans [8]. For example, one of the problems associated with using murine mAbs in humans is that they may induce an anti-mouse Ab response. Further, repeat administration of murine mAbs may result in rapid clearance of the murine mAbs and anaphylaxis, which can sometimes be fatal. To overcome this hurdle, the humanization of murine mAbs has been proposed, by which process murine Ab frameworks are replaced by human Ab ones in order to reduce immunogenicity of Abs in humans [9,10].

Thus, a need exists for a humanized anti-VEEV Ab.

### SUMMARY OF THE INVENTION

In one aspect, the present invention provides prophylaxis and post-exposure therapy against VEEV infection.

In one aspect, the invention provides a humanized rAb comprising a human immunoglobulin (Ig) framework and having grafted thereon complementarity determining regions (CDRs) from the murine mAb 1A4A1. In a preferred embodiment, the human 1g framework is obtained from IgG1.

In another aspect, the invention provides a humanized rAb having specificity to the E2 envelope protein of VEEV. More specifically, the rAb has specificity to the E2^{c} epitope of the E2 protein.

In another aspect, the invention provides a humanized rAb wherein the complementarity determining regions CDR1, CDR2 and CDR3 of the heavy chain variable region (VH) have the following amino acid sequences:

| | |
|---|---|
| CDRI: | SEQ ID NO: 1 |
| CDR2: | SEQ ID NO: 2 |
| CDR3:. | SEQ ID NO: 3 |

In another aspect, the invention provides a humanized rAb wherein the complementarity determining regions CDR1, CDR2 and CDR3 of the light chain variable region (VL) have the following amino acid sequences:

| | |
|---|---|
| CDR1: | SEQ ID NO: 4 |
| CDR2: | SEQ ID NO: 5 |
| CDR3:. | SEQ ID NO: 6 |

In a further aspect, the invention provides a humanized rAb having a VH comprising the amino acid sequence of SEQ ID NO: 7.

In a further aspect, the invention provides a humanized rAb having a VL comprising the amino acid sequence of SEQ ID NO: 8.

In another aspect, the invention provides a DNA sequence which encodes a polypeptide corresponding to a CDR grafted VH having the amino acid sequence according to SEQ ID NO: 7.

In another aspect, the invention provides a DNA sequence which encodes a polypeptide corresponding to a CDR grafted VL having the amino acid sequence according to SEQ ID NO: 8.

In a further aspect, the invention provides a DNA construct having a nucleic acid sequence according to SEQ ID NO:11 or SEQ ID NO:13.

In another aspect, the invention provides an expressed protein comprising a humanized rAb having an amino acid sequence according to SEQ ID NO: 12 or SEQ ID NO: 14.

The invention provides vectors containing such DNA sequences and host cells transformed thereby.

In other aspects, the invention provides methods and uses for treatment or prophylaxis of VEEV infection utilizing the rAbs described herein. The invention also provides pharmaceutical preparations for such treatment or prophylaxis.

### BRIEF DESCRIPTION OF THE DRAWINGS

These and other features of the invention will become more apparent in the following detailed description in which reference is made to the appended drawings wherein:

FIG. 1 is a representation of the external structure of the VEEV.

FIGS. 2a to 2d schematically illustrate murine, human, chimeric and humanized Abs, respectively.

FIGS. 3a to 3c schematically illustrate the humanization of the murine Ab variable region.

FIG. 4 schematically illustrates the cloning of the murine Ab VH and VL.

FIG. 5 schematically illustrates the humanization of the Ab VH and shows its amino acid sequence.

FIG. 6 schematically illustrates the humanization of the Ab VL and shows its amino acid sequence.

FIG. 7 schematically illustrates the design of a full Hu1A4A1IgG1 rAb gene in a single open reading frame with two versions, Hu1A4A1IgG1-furin and Hu1A4A1IgG1-2A.

FIG. 8 schematically illustrates the cloning of the Hu1A4A1IgG1-furin and Hu1A4A1IgG1-2A genes into an adenoviral vector respectively.

FIG. 9 schematically illustrates expression and purification of the Hu1A4A1IgG1-furin and Hu1A4A1IgG1-2A rAbs.

FIGS. 10 and 11 illustrate the results from the SDS-PAGE separation of the produced Hu1A4A1IgG1-furin rAb.

FIG. 12 illustrates the results from the sodium dodecyl sulfate-polyacrylamide gel electrophoresis (SDS-PAGE) separation of the produced Hu1A4A1IgG1-2A rAb.

FIG. 13 illustrates the results of the enzyme-linked immunosorbent assays (ELISA) for the reactivity of the Hu1A4A1IgG1-furin and Hu1A4A1IgG1-2A rAbs.

FIG. 14 schematically illustrates Hu1A4A1IgG1-2A was cleaved between the heavy and light chains as expected, whereas Hu1A4A1IgG-furin was not cleaved.

FIG. 15 schematically illustrates the neutralization assay used in assessing the neutralizing activity of the Hu1A4A1IgG1-furin and Hu1A4A1IgG1-2A rAbs against VEEV.

### DETAILED DESCRIPTION OF THE INVENTION

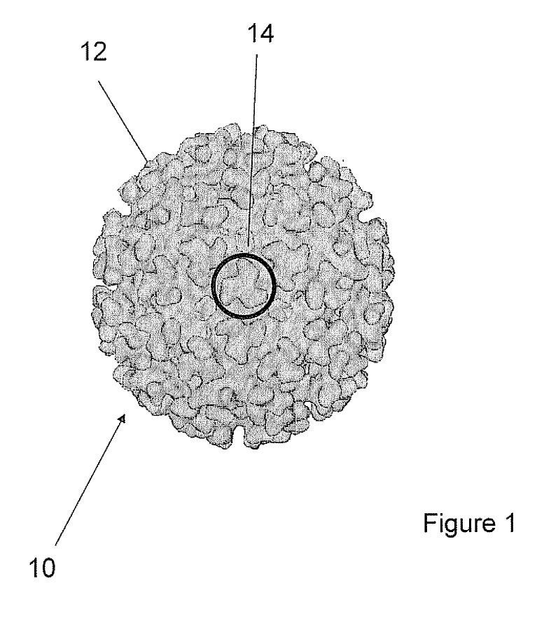

FIG. 1 illustrates the external structure of the VEEV. As shown, the virus 10 includes a nucleocapsid 12 enveloping the viral RNA genome. The envelope comprises glycoproteins E1 and E2, arranged in the form of heterodimers 14. Protein E2, which is responsible for viral attachment to the host cell, contains neutralizing epitopes.

As has been described in the prior art, the murine mAb 1A4A1 has been found to be specific to the VEEV E2 envelope protein and, further, has been found to have a strong neutralizing function against VEEV. The murine mAb, however, causes a sometimes fatal allergenic reaction in humans, resulting in the formation of human anti-mouse Abs (HAMA). It is for this reason that the present inventors have sought to humanize the 1A4A1 mAb so as to provide an effective agent to counter VEEV infection in humans.

In vivo efficacy studies in mice have demonstrated that treatment with murine mAb 1A4A1 leads to protection of animals from a lethal peripheral challenge with virulent VEEV. Thus, the present invention builds upon these findings by providing a humanized mAb 1A4A1 to reduce the foreignness of murine mAb in humans. For doing this, the majority of the non-human protein sequence (in one embodiment, more than 90%) of mAb 1A4A1 is replaced with a human Ab sequence and the resultant whole humanized mAb gene is then synthesized and cloned to an adenoviral vector. The recombinant adenoviral vector can be delivered as a therapeutic agent for prophylaxis or treatment of VEEV infection in humans. One advantage of this method is that the vector can express the humanized Ab in the human body for a long period of time. The humanized Ab can also be produced in cell culture and delivered directly as a therapeutic.

The humanization of the present anti-VEEV mAb 1A4A1 has not been done previously and particularly not for the prophylaxis or treatment of VEEV infection. The present invention provides in one embodiment a humanized Ab, referred to herein as Hu1A4A1IgG1, that retains the VEEV-binding specificity and neutralizing activity of murine 1A4A1 while not eliciting a HAMA response. As described further below, the humanized Ab comprises an Ig framework of human IgG1 and CDRs obtained from murine mAb 1A4A1. The rAb of the present invention is specific to an epitope of the E2 envelope glycoprotein of VEEV and, more specifically, to the E2^{c} epitope thereon.

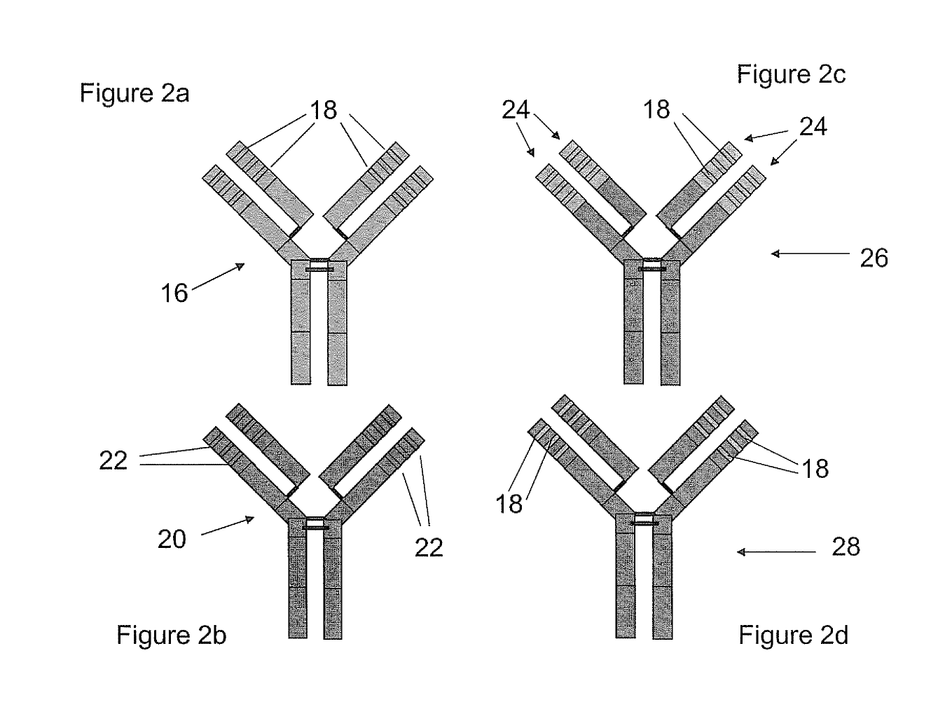

The construction of the humanized Ab of the invention is schematically illustrated in FIGS. 2a to 2d. FIG. 2a illustrates schematically the structure of a murine Ab 16 containing murine CDRs 18 on the respective variable regions. FIG. 2b shows a human Ab 20 containing human CDRs 22. As shown in FIG. 2c, a chimeric Ab 26 would comprise the murine variable regions 24, containing the murine CDRs 18, joined to the constant regions of the human Ab. On the other hand, FIG. 2d illustrates a humanized Ab 28 according to an embodiment of the invention, wherein only the murine CDRs 18 are grafted to the variable regions of the human Ab 20.

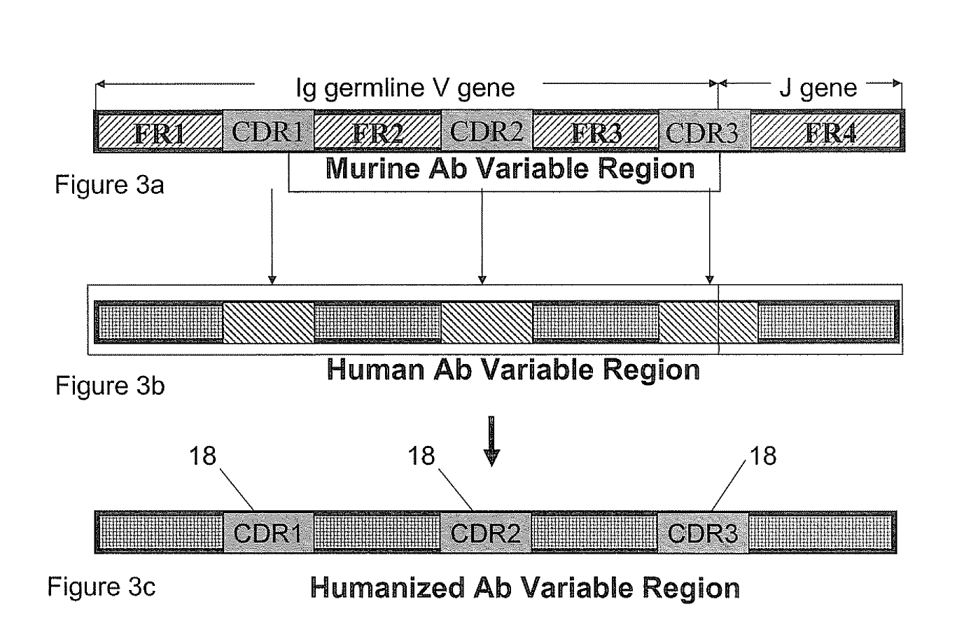

The substitution of the murine CDRs into the human Ig framework is illustrated also in FIGS. 3a to 3c. As shown, the humanized Ab variable region comprises the grafted CDRs, 18, from the murine Ab.

The protein sequences of the rAbs of the invention include linker sequences. The expressed rAbs of the invention have amino acid sequences as shown in SEQ ID NO:12 and SEQ ID NO:14. The nucleic acid constructs used in transfecting cells to express the above rAbs are shown in SEQ ID NO:11 and SEQ ID NO:13.

### EXAMPLES

The following examples are provided to illustrate embodiments of the present invention. The examples are not intended to limit the scope of the invention in any way.

### Example 1

### Construction of Hu1A4A1IgG1 and in vitro Studies

In the study described below, murine mAb 1A4A1 CDRs of VH, VL were grafted onto the frameworks of germline variable and joining (V, J) gene segments of human Ig heavy and light chains, respectively, which were chosen based on the CDR similarities between human Igs and murine mAb 1A4A1. Furthermore, the humanized VH and VL were, respectively, grafted onto human gamma 1 heavy chain constant regions (CHs) and kappa 1 light chain constant region (CL) to assemble the whole humanized Ab gene. The resultant whole humanized mAb gene was synthesized and cloned to an adenoviral vector. After the humanized Ab was expressed in HEK 293 cells and purified with protein L column, the Ab was demonstrated to retain antigen-binding specificity and neutralizing activity.

Materials and Methods

Humanization of Murine mAb 1A4A1

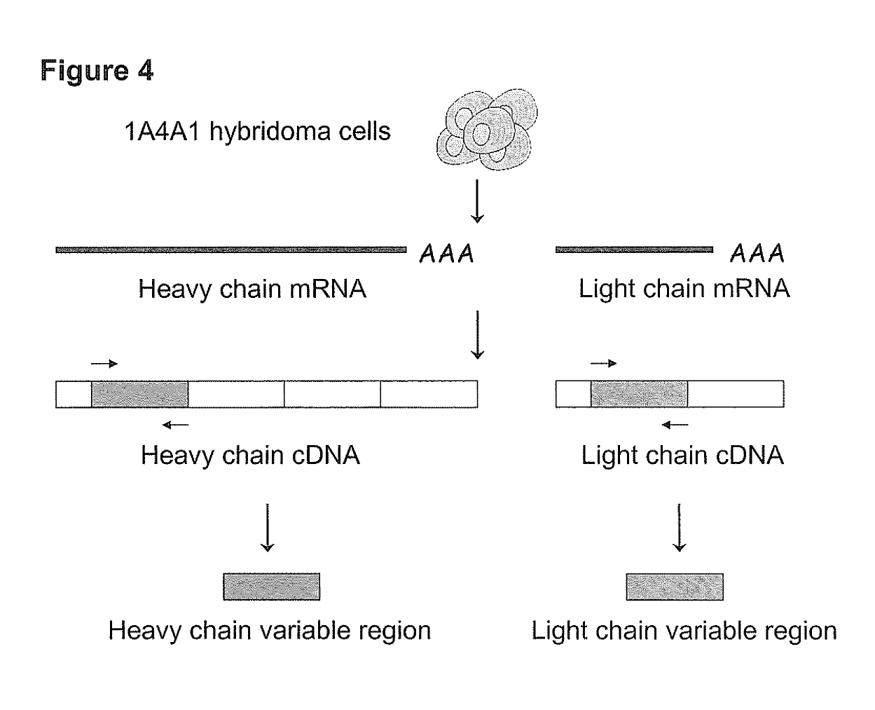

Murine mAb 1A4A1 was provided by Dr. J. T. Roehrig (Division of Vector-borne Infectious Diseases, Centers for Disease Control and Prevention, Fort Colins, Colo., USA). The VH and VL of mAb 1A4A1 were cloned in a single chain variable fragment (ScFv) format, mA116 previously [7], which showed to retain the same binding specificity as mAb 1A4A1 [11]. The humanization of VH and VL of murine mAb 1A4A1 was done by Absalus Inc. (Mountain View, Calif., USA). Briefly, in order to select human VH and VL frameworks 1-3, the VH and VL amino acid sequences of murine 1A4A1 were separately subjected to IgBlast and IMGT searches against the entire human Ig germline V gene segments and then human heavy and light chain germline V gene segments were selected based on their highest CDR 1 and 2 similarities with those of murine 1A4A1 VH and VL without consideration of framework similarity. Both human VH and VL framework 4 were selected, respectively, from human heavy and light chain J gene segments based on the highest similarities between human J gene segments and murine 1A4A1 VH and VL CDR3. Finally, CDRs of murine 1A4A1 VH and VL were, respectively, grafted onto the frameworks of selected germline V and J gene segments of human Ab heavy and light chains, resulting in humanized 1A4A1 (Hu1A4A1). Furthermore, the Hu1A4A1 VH and VL were, respectively, grafted onto human gamma 1 heavy chain CHs and kappa 1 light chain CL to assemble the whole humanized Ab gene, resulting in humanized 1A4A1IgG1 (Hu1A4A1IgG1). This process is illustrated in FIGS. 3 to 6.

Construction, Expression and Purification of Hu1A4A1IgG1 (Hu1A4A1IgG1-furin and Hu1A4A1IgG1-2A)

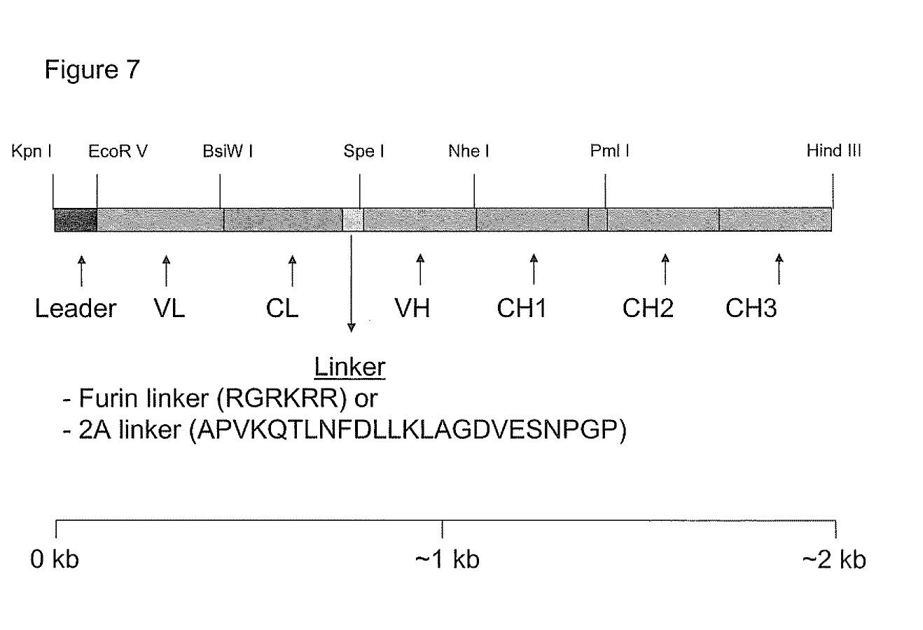

The Hu1A4A1IgG1 DNA sequence (˜2 kb) is schematically illustrated in FIG. 7. The nucleic acid sequence of the Hu1A4A1IgG1-furin rAb is provided in SEQ ID NO:11 and the nucleic acid sequence of the Hu1A4A1IgG1-2A rAb is provided in SEQ ID NO:13.

The Hu1A4A1IgG1 DNA sequences were synthesized as follows. As shown in FIG. 7, a light chain leader sequence was provided upstream from the light chain, followed by a furin or 2A linker (discussed further below) before the heavy chain. The whole DNA sequence flanked by Kpn I and Hind III was synthesized by GenScript Corporation (Scotch Plaines, N.J., USA) and cloned into pUC57 vector, resulting in pUC57-Hu1A4A1IgG1-furin or pUC57-Hu1A4A1IgG1-2A.

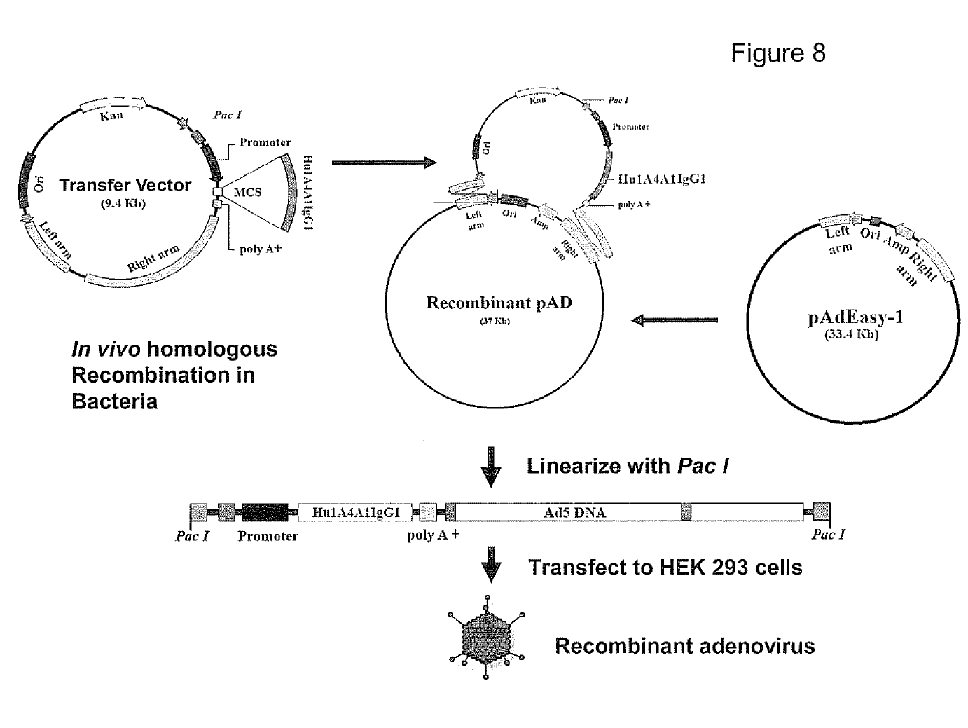

Recombinant adenovirus vectors expressing either Hu1A4A1IgG1-furin or Hu1A4A1IgG1-2A were constructed using AdEasy™ system (Qbiogene, Carlsbad, Calif., USA) according to the manufacturer's protocol. Briefly, the Kpn I-Hind III fragment of Hu1A4A1IgG1-furin or Hu1A4A1IgG1-2A was ligated to a Kpn I-Hind III-digested pShuttle-CMV vector. The resulting pShuttle construct was co-transformed with the pAdEasy-1 vector into Escherichia coli BJ5183 cells to produce recombinant adenoviral genomic constructs for Hu1A4A1IgG1-furin or Hu1A4A1IgG1-2A proteins. The recombinant adenoviral constructs, pAd-Hu1A4A1IgG1-furin and pAd-Hu1A4A1IgG1-2A were linearized with Pac I and transfected into HEK 293 cells (American Type Culture Collection, Manassas, Va., USA) cultured in Dulbecco's Modified Eagle's Medium supplemented with 5% fetal bovine serum (FBS) for amplification and then the amplified adenovirus was purified by a chromatographic method. This procedure is illustrated in FIG. 8.

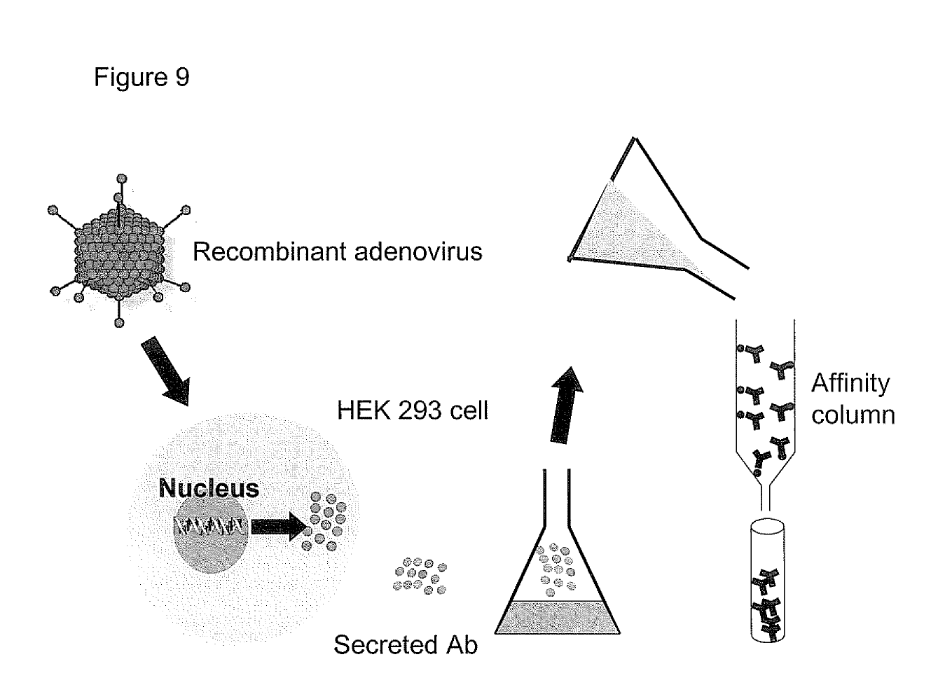

As illustrated in FIG. 9, the expression of Hu1A4A1IgG1-furin or Hu1A4A1IgG1-2A was achieved by first infecting HEK 293 cells with the recombinant adenovirus pAd-Hu1A4A1IgG1-furin or pAd-Hu1A4A1IgG1-2A at a multiplicity of infection (MOI) of 1. The infected cells were cultured for one week and the culture supernatant was harvested. The expressed Hu1A4A1IgG1-furin or Hu1A4A1IgG1-2A was purified using protein L agarose gel from Pierce (Brockville, Ont., Canada). Briefly, culture supernatant was dialyzed against phosphate buffer saline (PBS) (Sigma-Aldrich, Oakville, Ont., Canada) for 12 h and then concentrated using PEG (Sigma-Aldrich) to less than 50 ml. The concentrated sample was incubated with 2 ml protein L agarose gel at 4° C. for 1 h. The gel and supernatant mixture was then loaded to an empty column, which was subsequently washed with binding buffer. Bound Hu1A4A1IgG1-furin or Hu1A4A1IgG1-2A was eluted with elution buffer. The eluted Ab was further desalted using an excellulose column (Pierce) and then concentrated by a Centracon™ YM-30 (Millipore Corp., Bedford, Mass., USA).

The amino acid sequence of the expressed Hu1A4A1IgG1-furin is shown in SEQ ID NO:12 and the amino acid sequence of the expressed Hu1A4A1IgG1-2A is shown in SEQ ID NO:14.

SDS-PAGE

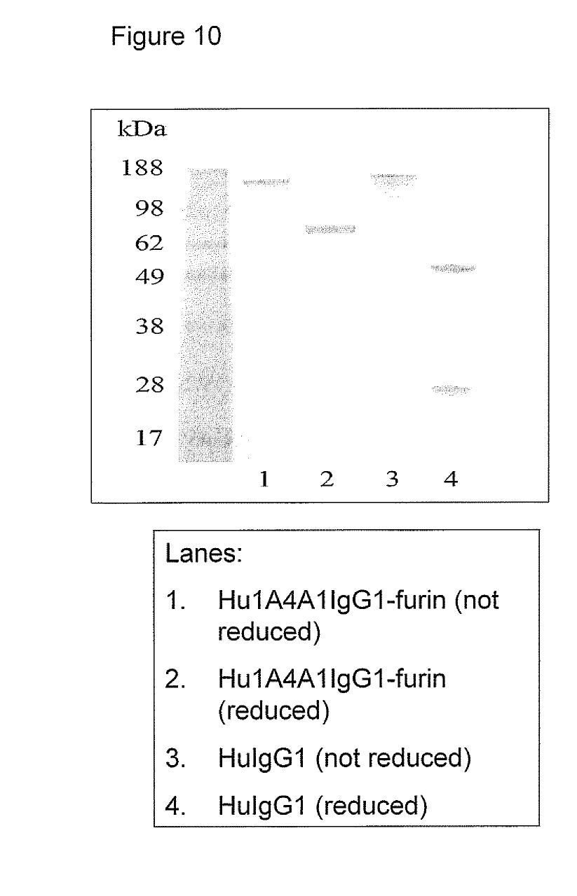

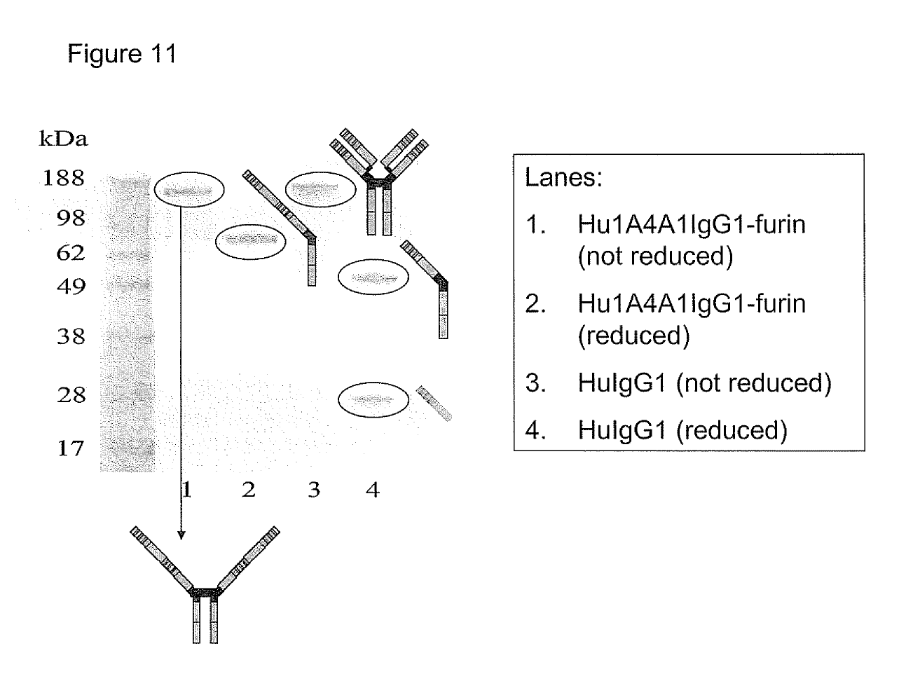

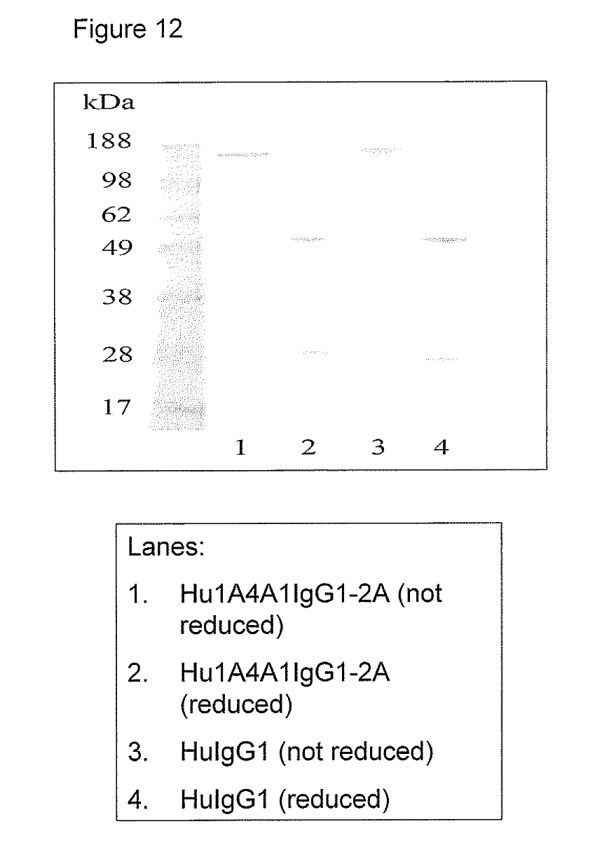

Abs were separated by 10% SDS-PAGE gels using a Mini-PROTEAN™ II apparatus (Bio-Rad Laboratories, Mississauga, Ont., Canada). The bands were visualized by SimplyBlue™ safestain staining (Invitrogen, Burlington, Ont., Canada). The molecular weights of the samples were estimated by comparison to the relative mobility values of standards of known molecular weights. The SDS-PAGE analyses of the purified Hu1A4A1IgG1-furin are illustrated in FIGS. 10 and 11. FIG. 12 illustrates the SDS-PAGE analysis of the purified Hu1A4A1IgG1-2A. As shown, lanes 1 and 3 correspond to purified Hu1A4A1IgG1 and control human IgG1 in a non-reducing condition and lanes 2 and 4 correspond to purified Hu1A4A1IgG1 and control human IgG1 in a reducing condition.

ELISA

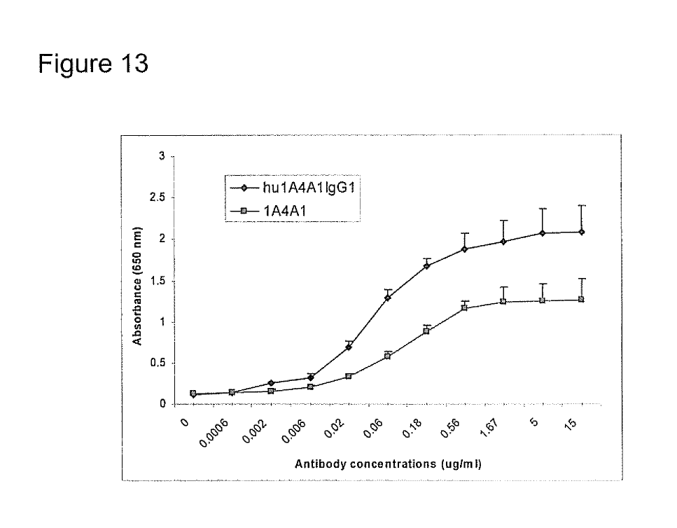

The reactivity of purified Hu1A4A1IgG1-furin or Hu1A4A1IgG1-2A to VEEV E2 antigen was determined by ELISA. Nunc Maxisorp™ flat bottomed 96-well plates (Canadian Life Technologies, Burlington, Ont., Canada) were coated overnight at 4° C. with recombinant VEEV E2 antigen at a concentration of 10 μg/ml in carbonate bicarbonate buffer, pH 9.6. The plates were washed five times with PBS containing 0.1% Tween™-20 (PBST) and then blocked in 2% bovine serum albumin for 2 h at room temperature. After five washes with PBST, the plates were incubated for 2 h at room temperature with various concentrations of Hu1A4A1IgG1-furin, Hu1A4A1IgG1-2A or 1A4A1 Abs diluted in PBST. Following five washes with PBST, the plates were incubated for 2 h at room temperature with horseradish peroxidase (HRP)-conjugated rabbit anti-human IgG fragment crystallizable portion or HRP-conjugated rabbit anti-mouse IgG (Jackson ImmunoResearch Laboratories Inc., West Grove, Pa., USA) diluted 1:5000 in PBST. Finally, the plates were washed five times with PBST and developed for 10 min at room temperature with a 3,3′,5,5′-tetramethylbenzidine substrate (Kirkegaard and Perry Laboratories). The reactions were read at an absorbance of 650 nm by a microplate autoreader (Molecular Devices, Sunnyvale, Calif., USA). The results of the ELISA Hu1A4A1IgG1-antigen binding assay are illustrated in FIG. 13.

Neutralization Assay in Vitro

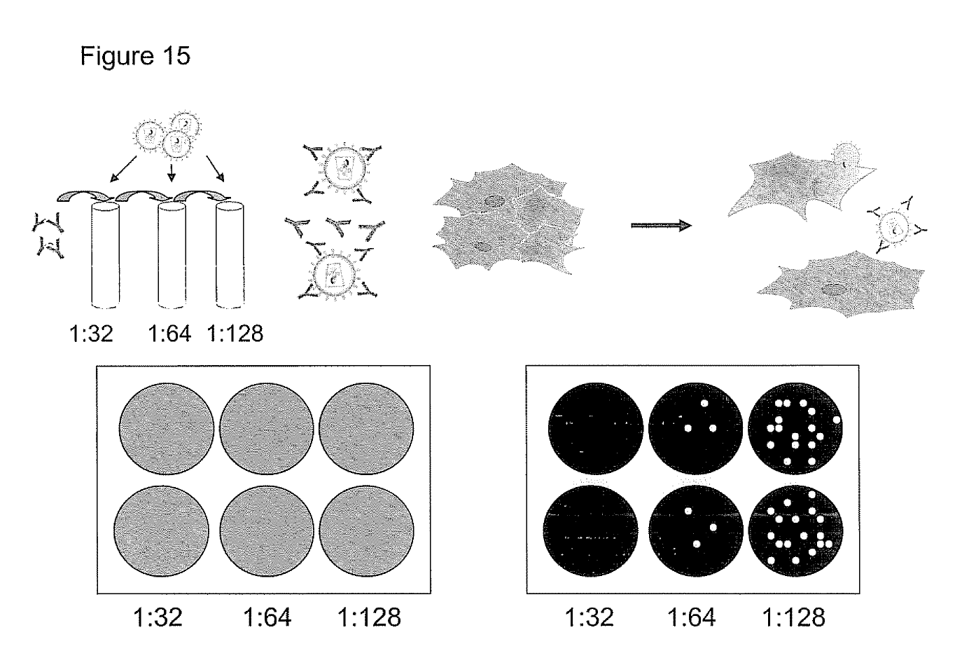

Neutralizing activity of each of Hu1A4A1IgG1-furin and Hu1A4A1IgG1-2A against VEEV (strain TC-83) was analyzed by a plague reduction assay. Briefly, each Ab was serially two-fold diluted and mixed with an equal volume containing 50 plaque-forming units of virus per 100 μl. After mixtures were incubated for 1 h at room temperature, 200 μl of the mixture was inoculated in duplicate into wells of six-well plates containing confluent Vero cell monolayers and incubated at 37° C. for 1 h. At the end of the incubation, the virus/Ab mixtures were removed from the wells before the wells were overlaid by tragacanth gum and then incubated for 2 days. The wells were stained with 0.3% crystal violet and plaques were counted. Neutralization titre was expressed as the highest Ab dilution that inhibited 50% of virus plaques. This procedure is illustrated in FIG. 15.

Results and Discussion

Different approaches have been developed to humanize murine Abs in order to reduce the antigenicity of murine Abs in humans [9,10]. One widely used approach is CDR-grafting, which involves the grafting of all murine CDRs onto a human Ab frameworks. The human Ab frameworks are chosen based on their similarities to the frameworks of the murine Ab to be humanized. The CDR-grafting approach has been proven successful in some cases. However, in many more instances, this humanization process could result in CDR conformation changes, which affect the antigen-binding affinity. To restore the affinity, additional work for back-mutation of several murine framework amino acids, which are deemed to be critical for CDR loop conformation, have to be done.

Recently, Hwang et al. [12] employed an approach which consisted of grafting CDRs onto human germline Ab frameworks based on the CDR sequence similarities between the murine and human Abs while basically ignoring the frameworks. Because the selection of the human frameworks is driven by the sequence of the CDRs, this strategy minimizes the differences between the murine and human CDRs. This approach has the potential to generate humanized Abs that retain their binding affinity to their cognate antigen. Further, since all residues in frameworks are from human Ab germline sequences, the potential immunogenicity of non-human Abs is highly reduced.

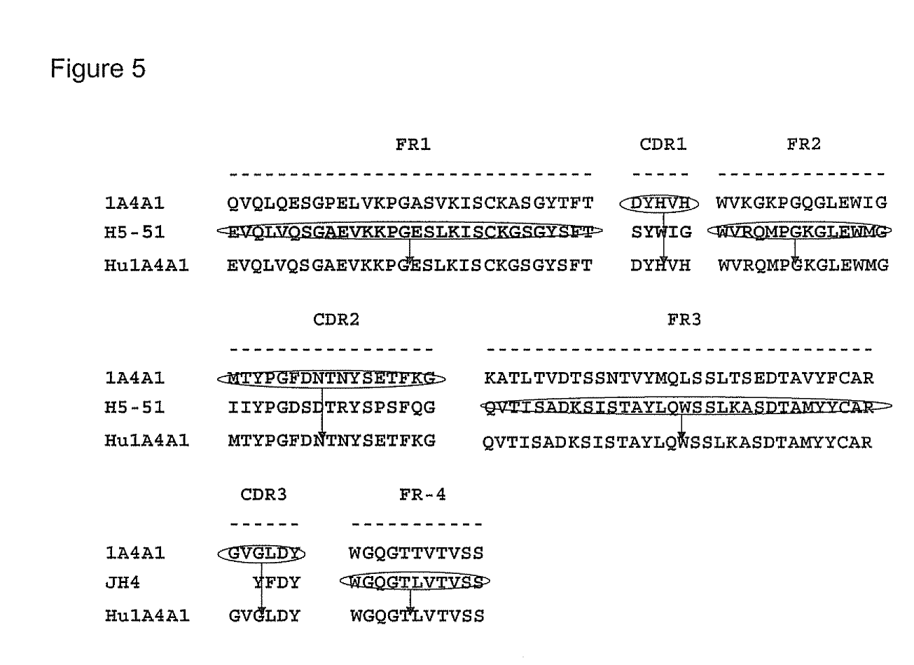

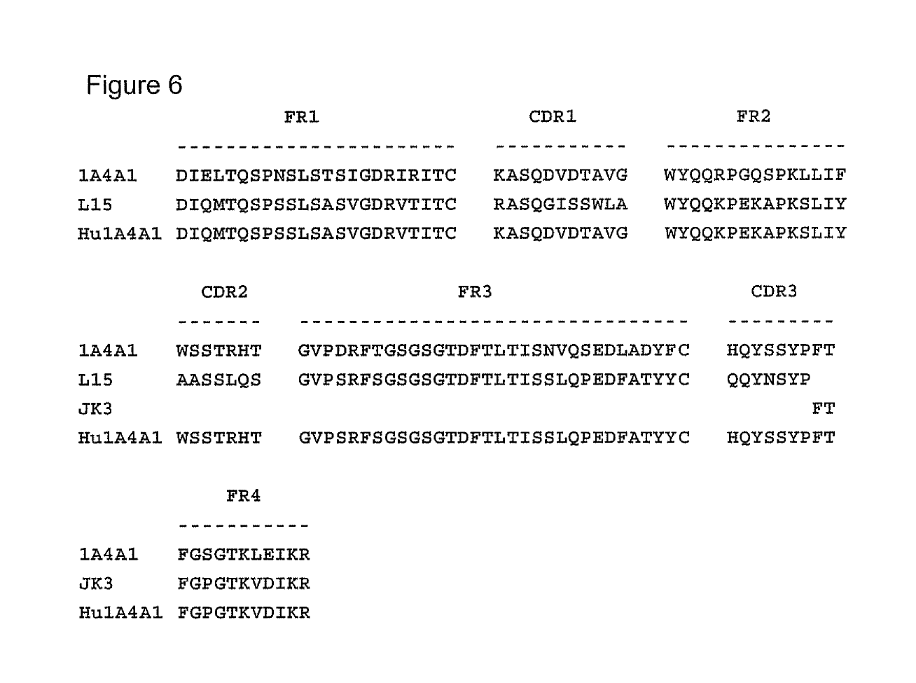

Using the above approach, and as disclosed herein, the present inventors humanized an anti-VEEV murine mAb 1A4A1. The amino acid sequences of VH and VL from murine 1A4A1 were first aligned with human Ig germline V and J genes. As shown in FIG. 5, the human heavy chain V gene segment H5-51 and J gene segment JH4 were selected to provide the frameworks for the murine 1A4A1 VH. Similarly, as shown in FIG. 6, for the murine 1A4A1 VL, the human light chain V gene segment L15 and J gene segment Jk3 were selected.

The identities of the CDR1 and CDR2 amino acid sequences between murine 1A4A1 VH and the human H5-51 gene segment were 20% and 47%, respectively, while the identity of the CDR3 between murine 1A4A1 VH and the JH4 gene segment was 33%. For the light chain, the identities of the CDR1 and CDR2 between murine 1A4A1 VL and the human L15 gene segment were 27% and 14%, respectively, while the identity of the CDR3 between murine 1A4A1 VL and human Jk3 gene segment was 22%. The CDRs of murine 1A4A1 VH were then grafted onto the frameworks of selected human Ig germline H5-51 and JH4 gene segments, while the CDRs of murine 1A4A1 VL were grafted onto human L15 and Jk3 gene segments. The hu1A4A1 VH was further grafted onto the human gamma 1 heavy chain CHs to form a complete heavy chain, while the VL was grafted onto the human kappa 1 light chain CL to form a whole humanized light chain. This procedure is schematically illustrated in FIGS. 5 and 6 with the end structure being illustrated in FIG. 7.

As shown in FIG. 5, the murine 1A4A1 VH CDRs grafted onto the human framework comprised the following amino acid sequences:

| | |
|---|---|
| VH ODR1: DYHVH | (SEQ ID NO: 1) |
| VH CDR2: MTYPGFDNTNYSETFKG | (SEQ ID NO: 2) |
| VH CDR3: GVGLDY | (SEQ ID NO: 3) |

As shown in FIG. 6, the murine 1A4A1 VL CDRs grafted onto the human framework comprised the following amino acid sequences:

| | |
|---|---|
| VL CDR1: KASQDVDTAVG | (SEQ ID NO: 4) |
| VL CDR2: WSSTRHT | (SEQ ID NO: 5) |
| VL CDR3: HQYSSYPFT | (SEQ ID NO: 6) |

As shown in FIG. 5, the VH of the humanized Ab according to the present invention comprises the following amino acid sequence:

Hu-VH:

(SEQ ID NO: 7)

EVQLVQSGAEVKKPGESLKISCKGSGYSFTDYHVHWVRQMPGKGLEWMGM
TYPGFDNTNYSETFKGQVTISADKSISTAYLQWSSLKASDTAMYYCARGV
GLDYWGQGTLVTVSS.

Thus, as shown in FIG. 6, the VL of the humanized Ab according to the present invention comprises the following amino acid sequence:

Hu-VL:

(SEQ ID NO: 8)

DIQMTQSPSSLSASVGDRVTITCKASQDVDTAVGWYQQKPEKAPKSLIYW
SSTRHTGVPSRFSGSGSGTDFTLTISSLQPEDFATYYCHQYSSYPFTFGP
GTKVDIKR.

In order to express heavy and light chains in a monocistronic construct, a six-residue peptide, RGRKRR (SEQ ID NO: 9) containing the recognition site for the protease furin, designated as “furin linker”, or a twenty-four-residue peptide of the foot-and-mouth-disease virus (FMDV)-derived 2A self-processing sequence, APVKQTLNFDLLKLAGDVESNPGP (SEQ ID NO: 10), designated as “2A linker”, was incorporated between the two chains. The location of the furin or 2A linker within the nucleic acid constructs of the Abs is illustrated in FIG. 7. Furin is a ubiquitous subtilisin-like proprotein convertase, which is the major processing enzyme of the secretory pathway [13]. The furin minimal cleavage site is R-X-X-R; however, the enzyme prefers the site R-X-(K/R)-R. An additional R at the P6 position appears to enhance cleavage. The FMDV-derived 2A linker is able to cleave at its own C terminus between the last two residues through an enzyme-independent but undefined mechanism, probably by ribosomal skip, during protein translation. To get the expressed Ab to be secreted to culture media, a leader sequence was added upstream to the Ab gene. FIG. 7 illustrates the synthesized DNA sequence, of approximately 2 kb, including the human Ab kappa light chain L15 leader sequence, the humanized light chain (VL+CL), the furin or 2A linker, and the humanized heavy chain (VH+CH1+CH2+CH3). This sequence was then cloned into an adenoviral vector. The unique restriction sites, as also shown in FIG. 7, flanking the V regions, which allow for efficient V region replacement and at the heavy chain V-C region junction for generation of fragment antigen-binding portion of Ab (Fab), were also designed.

Protein G and A columns are widely used for a quick purification for Abs because of protein G and A binding to the Fc portion of Ig. However, protein G and A cannot only bind to human Ig, but also bind to bovine Ig, therefore they cannot be used for purification of Hu1A4A1IgG1-furin or Hu1A4A1IgG1-2A in our study since pAd-Hu1A4A1IgG1-furin or pAd-Hu1A4A1IgG1-2A-infected HEK 293 cells were cultured in the medium with 5% FBS containing a high percentage of bovine Ig. Unlike protein G and A, protein L binds Ig through interactions with the light chains. Protein L only binds to Ig containing light chains of type kappa 1, 3 and 4 in human and kappa 1 in mouse. Most importantly, protein L does not bind to bovine Ig. Since our humanized Ab has human kappa 1 chain, we chose a protein L column to purify Hu1A4A1IgG1-furin or Hu1A4A1IgG1-2A to eliminate co-purification of bovine Ig. In this way, the purity of Hu1A4A1IgG1-furin or Hu1A4A1IgG1-2A was relatively high in SDS-PAGE as shown in FIGS. 10, 11 and 12.

When the purified product was subjected to 10% SDS-PAGE, Hu1A4A1IgG1-furin and Hu1A4A1IgG1-2 showed up in a different way. As illustrated in FIG. 12, Hu1A4A1IgG1-2A showed the same patterns as a control human IgG1, one band of ˜150 kDa in non-reducing condition (intact disulfide bridges) and two bands, 50 kDa for heavy chains and 25 kDa for light chains (broken disulfide bridges) in reducing condition, indicating that the 2A linker underwent self-processing perfectly. On the other hand, Hu1A4A1IgG1-furin showed only one clear band of ˜75 kDa in reducing condition observed as illustrated in FIGS. 10 and 11, indicating that the furin linker was not cleaved. However, in another study (data not shown), the same furin linker sequence was cleaved in another Fab construct expressed in a mammalian system. This indicated the conformation of expressed Hu1A4A1IgG1-furin probably rendered the furin linker inaccessible to furin or that the sequence surrounding the furin linker influenced furin cleavage.

The specific binding reactivities of purified Hu1A4A1IgG1-furin and Hu1A4A1IgG1-2A to VEEV E2 antigen were examined by ELISA. As illustrated in FIG. 13, both versions of the Hu1A4A1IgG1 were found to bind to VEEV E2 in a dose-dependent manner, similar to the binding to VEEV E2 of its parental murine 1A4A1, indicating this non-cleaved Ab was still reactive to VEEV E2 antigen in ELISA. Furthermore, both versions were evaluated for their ability to block VEEV infection in Vero cells using a standard plaque-reduction assay. The Hu1A4A1IgG1-furin showed a neutralizing activity with 50% plaque reduction neutralization titer at 0.78 μg/ml, whereas Hu1A4A1IgG1-2A showed a much higher neutralization titre at 0.1 μg/ml.

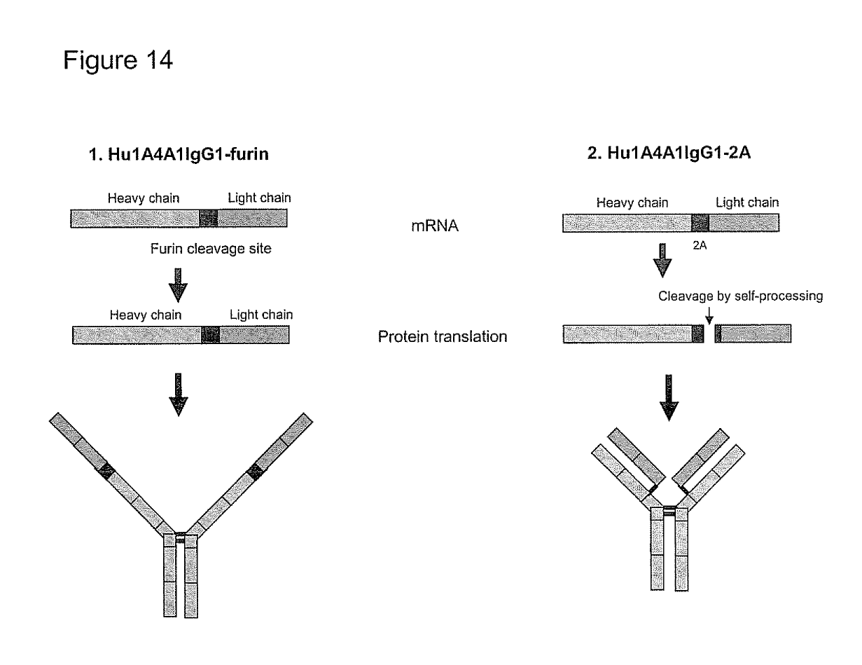

From the above results, it is concluded that the murine 1A4A1 Ab was successfully humanized. As illustrated in FIG. 14, the expressed and purified Ab of Hu1A4A1IgG1-2A was cleaved between the heavy and light chains as expected; however, Hu1A4A1IgG1-furin was not cleaved. Nevertheless, the present inventors have exhibited that both versions of the Hu1A4A1IgG1 retained the antigen binding specificity and virus neutralizing activity. Thus, the Hu1A4A1IgG1-furin or Hu1A4A1IgG1-2A discussed and characterized herein would serve as an effective prophylactic and therapeutic agent against VEEV infection.

### Example 2

### In vivo Study—Protection of Mice from VEEV Challenge by Passive Immunization with Hu1A4A1IgG1-furin or Hu1A4A1IgG1-2A

Materials and Methods

Passive Immunization

Balb/c mice aged 6-8 weeks were injected intraperitoneally (i.p) with 50 μg of Hu1A4A1IgG1-furin or Hu1A4A1IgG1-2A in 100 μl PBS, human anti-VEEV IgG in 100 μl PBS (positive control) or 100 μl PBS alone (negative control) 24 h prior to VEEV challenge.

VEEV Challenge

Each mouse was challenged subcutaneously (s.c.) with 30-50 plaque forming units (pfu) of virulent VEEV (Trinidad donkey, TRD) in 50 μl of Leibovitz L15 maintenance medium (L15MM) 24 h after passive immunization. The challenge dose approximated to 100×50% lethal dose (LD50). Mice were examined frequently for signs of illness for 14 days, and humane endpoints were used.

Results

Hu1A4A1IgG1-furin or Hu1A4A1IgG1-2A Clearance in Mice

To determine the half-life of Hu1A4A1IgG1-furin or Hu1A4A1IgG1-2A in mouse serum, groups of 4 mice, were injected i.p. with 50 μg, each mouse, of either Hu1A4A1IgG1-furin or Hu1A4A1IgG1-2A, or human anti-VEEV IgG and bled from the vein at increasing time intervals after injection. The quantity of Ab present in serum samples was estimated by immunoassay. Hu1A4A1IgG1-furin or Hu1A4A1IgG1-2A had a similar half-life as human anti-VEEV IgG, around 10 days.

Protection of Mice from VEEV Challenge by Passive Immunization with Hu1A4A1IgG1-Furin or Hu1A4A1IgG1-2A

Groups of 8 mice were injected i.p. with the Hu1A4A1IgG1-furin, Hu1A4A1IgG1-2A, human anti-VEEV IgG or PBS alone and 24 h later challenged s.c. with 100×LD50 of VEEV. None of the PBS alone treated mice survived. All the Hu1A4A1IgG1-furin or Hu1A4A1IgG1-2A treated mice survived the VEEV challenge without any clinical signs at 14 days post-challenge.

Discussion

Passive immunization of the Hu1A4A1IgG1-furin or Hu1A4A1IgG1-2A in mice (50 pg/mouse) 24 h before virulent VEEV challenge provided 100% protection against 100×LD50 challenge of VEEV when mice were treated with 50 μg/each mouse of Hu1A4A1IgG1-furin or Hu1A4A1IgG1-2A. The mice were also found to be asymptomatic throughout the 14 day observation period. These results indicate that the humanized anti-VEEV rAbs of the present invention has prophylactic capacity against VEEV infections. The half-lives of the humanized anti-VEEV rAbs in mice was around 10 days suggesting that the humanized anti-VEEV rAbs of the invention would be an effective prophylactic against VEEV for at least several weeks.

Bibliography

One or more of the following documents have been referred to in the present disclosure. The following documents are incorporated herein by reference in their entirety.

[1] Weaver S C, Ferro C, Barrera F, Boshell J, Navarro J C. Venezuelan equine encephalitis. Annu Rev Entomol 2004;49:141-74.

[2] Rivas F, Diaz L A, Cardenas V M, Daza E, Bruzon L, Alcala A, et al. Epidemic Venezuelan equine encephalitis in La Guajira, Colombia, 1995. J Infect Dis 1997;175:828-32.

[3] Pittman P R, Makuch R S, Mangiafico J A, Cannon T L, Gibbs P H, Peters C J. Long-term duration of detectable neutralizing antibodies after administration of live-attenuated VEE vaccine and following booster vaccination with inactivated VEE vaccine. Vaccine 1996; 14:337-43.

[4] Jahrling P B, Stephenson E H. Protective efficacies of live attenuated and formaldehyde-inactivated Venezuelan equine encephalitis virus vaccines against aerosol challenge in hamsters. J Clin Microbiol 1984; 19:429-31.

[5] France J K, Wyrick B C, Trent D W. Biochemical and antigenic comparison of the envelope glycoproteins of Venezuelan equine encephalomyelitis virus strains. J Gen Virol 1979; 44:725-40.

[6] Roehrig J T, Day J W, Kinney R M. Antigenic analysis of the surface glycoproteins of a Venezuelan equine encephalomyelitis virus (TC-83) using monoclonal antibodies. Virology 1982;118:269-78.

[7] Roehrig J T, Mathews J H. The neutralization site on the E2 glycoprotein of Venezuelan equine encephalomyelitis (TC-83) virus is composed of multiple conformationally stable epitopes. Virology 1985; 142:347-56.

[8] Schroff R W, Foon K A, Beatty S M, Oldham R K, Morgan Jr A C. Human anti-murine immunoglobulin responses in patients receiving monoclonal antibody therapy. Cancer Res 1985; 45:879-85.

[9] Verhoeyen M, Milstein C, Winter G. Reshaping human antibodies: grafting an antilysozyme activity. Science 1988; 239:1534-6.

[10] Dall'Acqua W F, Damschroder M M, Zhang J, Woods R M, Widjaja L, Yu J, et al. Antibody humanization by framework shuffling. Methods 2005; 36:43-60.

[11] Hu W G, Alvi A Z, Fulton R E, Suresh M R, Nagata L E. Genetic engineering of streptavidin-binding peptide tagged single-chain variable fragment antibody to Venezuelan equine encephalitis virus. Hybrid Hybridomics 2002; 21:415-20.

[12] Hwang W Y, Almagro J C, Buss T N, Tan P, Foote J. Use of human germline genes in a CDR homology-based approach to antibody humanization. Methods 2005; 36:35-42.

[13] van den Ouweland A M, van Duijnhoven H L, Keizer G D, Dorssers L C, Van de Ven W J. Structural homology between the human fur gene product and the subtilisin-like protease encoded by yeast KEX2. Nucleic Acids Res 1990; 18:664.

[14] Fulton R E, Nagata, L, Alvi, A; U.S. Pat. No. 6,818,748, Nov. 16, 2004.

[15] Johnson K M, Martin D H. Venezuelan equine encephalitis. Adv. Vet Sci Comp Med. 1974; 18(0):79-116.

[16] Groot H, (1972) The health and economic importance of Venezuelan equine encephalitis (VEE) in Venezuelan encephalitis, Scientific publication no. 243, pp. 7-16, Pan American Health Organization, Washington D.C.

[17] Phillpotts R J, Jones L D, Howard S C, Monoclonal antibody protects mice against infection and disease when given either before or up to 24 h after airborne challenge with virulent Venezuelan equine encephalitis virus. Vaccine, Feb. 22, 2002; 20 (11-12); 1497-504.

Although the invention has been described with reference to certain specific embodiments, various modifications thereof will be apparent to those skilled in the art without departing from the purpose and scope of the invention as outlined in the claims appended hereto. Any examples provided herein are included solely for the purpose of illustrating the invention and are not intended to limit the invention in any way. Any drawings provided herein are solely for the purpose of illustrating various aspects of the invention and are not intended to be drawn to scale or to limit the invention in any way. The disclosures of all prior art recited herein are incorporated herein by reference in their entirety.

1. A humanized rAb comprising a human lg framework and having grafted thereon complementarity determining regions, CDRs, from the murine mAb 1A4A1.

2. The rAb of claim 1 wherein said rAb has specificity to VEEV.

3. The rAb of claim 2 wherein said rAb has specificity to an epitope of the E2 envelope protein of VEEV.

4. The rAb of claim 3 wherein said epitope is E2^{c}.

5. The humanized rAb of claim 1 having a VH with complementarity determining regions COR1, CDR2 and CDR3 having the following amino acid sequences:

| | |
|---|---|
| CDR1: | SEQ ID NO: 1 |
| CDR2: | SEQ ID NO: 2 |
| CDR3:. | SEQ ID NO: 3 |

6. The humanized rAb of claim 1 having a VL with complementarity determining regions COR1, CDR2 and CDR3 having the following amino acid sequences:

| | |
|---|---|
| CDR1: | SEQ ID NO: 4 |
| CDR2: | SEQ ID NO: 5 |
| CDR3:. | SEQ ID NO: 6 |

7. The humanized rAb of claim 1 having a VH comprising an amino acid sequence according to SEQ ID NO: 7.

8. The humanized rAb of claim 1 having a VL comprising an amino acid sequence according to SEQ ID NO: 8.

9. The use of the rAb of claim 1 for the treatment or prophylaxis of VEEV infection.

10. A pharmaceutical preparation comprising as the active ingredient a humanized rAb as claimed in claim 1 or a fragment thereof and a pharmaceutically acceptable carrier or diluent.

11. A DNA sequence which encodes a polypeptide corresponding to a CDR grafted VH having an amino acid sequence according to SEQ ID NO: 7.

12. A DNA sequence which encodes a polypeptide corresponding to a CDR grafted VL having an amino acid sequence according to SEQ ID NO: 8.

13. A cloning or expression vector containing a DNA sequence which encodes a polypeptide corresponding to a CDR grafted VH having an amino acid sequence according to SEQ ID NO: 7 or a CDR grafted VL having an amino acid sequence according to SEQ ID NO: 8.

14. A host cell transformed with a cloning or expression vector according to claim 13.

15. A method of treatment or prophylaxis against VEEV infection in a mammal comprising administering to said mammal the rAb according to claim 1.

16. The humanized rAb of claim 1 wherein said rAb has an amino acid sequence according to SEQ ID NO:12 or SEQ ID NO:14.

17. A nucleic acid sequence encoding a humanized rAb comprising a human lg framework and having grafted thereon CDRs from the murine mAb 1A4A1, said nucleic acid sequence comprising SEQ ID NO:11 or SEQ ID NO:13.

18. A cloning or expression vector containing a DNA sequence according to claim 17.

19. A host cell transformed with a cloning or expression vector according to claim 18.

20. A method of treatment or prophylaxis against VEEV infection in a mammal comprising administering to said mammal the rAb according to claim 16.


SEQUENCE LISTING (14 sequences)

SEQ ID NO: 1 - protein, 5 aa, mouse
DYHVH

SEQ ID NO: 2 - protein, 17 aa, mouse
MTYPGFDNTNYSETFKG

SEQ ID NO: 3 - protein, 6 aa, mouse
GVGLDY

SEQ ID NO: 4 - protein, 11 aa, mouse
KASQDVDTAVG

SEQ ID NO: 5 - protein, 7 aa, mouse
WSSTRHT

SEQ ID NO: 6 - protein, 9 aa, mouse
HQYSSYPFT

SEQ ID NO: 7 - protein, 115 aa, Artificial
  Humanized VH region with grafted 1A4A1 CDRs
EVQLVQSGAEVKKPGESLKISCKGSGYSFTDYHVHWVRQMPGKGLEWMGMTYPGFDNTNYSETFKGQVTISADKSISTAYLQWSSLKASDTAMYYCARGVGLDYWGQGTLVTVSS

SEQ ID NO: 8 - protein, 108 aa, Artificial
  Humanized VL region with grafted 1A4A1 CDRs
DIQMTQSPSSLSASVGDRVTITCKASQDVDTAVGWYQQKPEKAPKSLIYWSSTRHTGVPSRFSGSGSGTDFTLTISSLQPEDFATYYCHQYSSYPFTFGPGTKVDIKR

SEQ ID NO: 9 - protein, 6 aa, Artificial
  Furin recognition site (furin linker)
RGRKRR

SEQ ID NO: 10 - protein, 24 aa, Artificial
  FMDV self processing sequence (2A linker)
APVKQTLNFDLLKLAGDVESNPGP

SEQ ID NO: 11 - DNA, 2070 bp, Artificial
  Hu1A4A1IgG1-Furin DNA Sequence
atggacatgagggtcctcgctcagctcctggggctcctgctgctctgtttcccaggtgccagatgtgatatccagatgacccagtctccatcctcactgtctgcatctgtaggagacagagtcaccatcacttgtaaggccagccaggacgtggacaccgccgtgggctggtatcagcagaaaccagagaaagcccctaagtccctgatctattggagcagcacccggcacaccggggtcccatcaaggttcagcggcagtggatctgggacagatttcactctcaccatcagcagcctgcagcctgaagattttgcaacttattactgccaccagtacagcagctaccccttcaccttcggccctgggaccaaagtggacatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttctggtcgtggacgtaagagaagagaggtgcaactagtgcagtctggagcagaggtgaaaaagcccggggagtctctgaagatctcctgtaagggttctggatacagctttaccgactaccatgtgcactgggtgcgccagatgcccgggaaaggcctggagtggatggggatgacctaccccggcttcgacaacaccaactacagcgagaccttcaagggccaggtcaccatctcagccgacaagtccatcagcaccgcctacctgcagtggagcagcctgaaggcctcggacaccgccatgtattactgtgcgagaggcgtgggcctggactactggggccaaggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacgtgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 12 - protein, 689 aa, Artificial
  Hu1A4A1IgG1-Furin Amino Acid Sequence
MDMRVLAQLLGLLLLCFPGARCDIQMTQSPSSLSASVGDRVTITCKASQDVDTAVGWYQQKPEKAPKSLIYWSSTRHTGVPSRFSGSGSGTDFTLTISSLQPEDFATYYCHQYSSYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGRGRKRREVQLVQSGAEVKKPGESLKISCKGSGYSFTDYHVHWVRQMPGKGLEWMGMTYPGFDNTNYSETFKGQVTISADKSISTAYLQWSSLKASDTAMYYCARGVGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 2175 bp, Artificial
  Hu1A4A1IgG1-2A DNA Sequence
atggacatgagggtcctcgctcagctcctggggctcctgctgctctgtttcccaggtgccagatgtgatatccagatgacccagtctccatcctcactgtctgcatctgtaggagacagagtcaccatcacttgtaaggccagccaggacgtggacaccgccgtgggctggtatcagcagaaaccagagaaagcccctaagtccctgatctattggagcagcacccggcacaccggggtcccatcaaggttcagcggcagtggatctgggacagatttcactctcaccatcagcagcctgcagcctgaagattttgcaacttattactgccaccagtacagcagctaccccttcaccttcggccctgggaccaaagtggacatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtgcaccggtgaaacagactttgaattttgaccttctcaagttggcgggagacgtcgagtccaaccctgggcccatggggtcaaccgccatcctcgccctcctcctggctgttctccaaggagtctgttccgaggtgcaactagtgcagtctggagcagaggtgaaaaagcccggggagtctctgaagatctcctgtaagggttctggatacagctttaccgactaccatgtgcactgggtgcgccagatgcccgggaaaggcctggagtggatggggatgacctaccccggcttcgacaacaccaactacagcgagaccttcaagggccaggtcaccatctcagccgacaagtccatcagcaccgcctacctgcagtggagcagcctgaaggcctcggacaccgccatgtattactgtgcgagaggcgtgggcctggactactggggccaaggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacgtgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 14 - protein, 724 aa, Artificial [sequence omitted]